(19) United States
(12) Patent Application Publication
(10) Pub. No.: US 2010/0262029 A1
(43) Pub. Date: Oct. 14, 2010

(54) NEEDLE IMPLANTABLE ATRIAL FIBRILLATION MONITOR AND METHODS FOR USE THEREWITH

(76) Inventors: N. Patrick Kelly, Austin, TX (US); Antonio Torrini, Austin, TX (US); Daniel L. Ray, Austin, TX (US)

Correspondence Address: GARLICK HARRISON & MARKISON, P.O. BOX 160727, AUSTIN, TX, 78716-0727, US

(21) Appl. No.: 12/423,407

(22) Filed: Apr. 14, 2009

(51) Int. Cl. A61B 5/046

(52) U.S. Cl. 600/518

(57) ABSTRACT

A needle implantable atrial fibrillation monitor includes a plurality of terminals that generate a terminal voltage when the needle implantable atrial fibrillation monitor is implanted in a patient's body. An analog processing circuit generates a monitoring signal based on the terminal voltage. An analog to digital converter converts the monitoring signal to monitoring data. A processor analyzes the monitoring data, generates events and compresses the event data and that generates compressed data in response thereto. A memory stores the compressed data. An RF interface transmits the compressed data to a base station via RF signaling when the needle implantable atrial fibrillation monitor is implanted in the patient's body.

### CROSS REFERENCE TO RELATED PATENTS

NOT APPLICABLE

### STATEMENT REGARDING FEDERALLY SPONSORED RESEARCH OR DEVELOPMENT

NOT APPLICABLE

### INCORPORATION-BY-REFERENCE OF MATERIAL SUBMITTED ON A COMPACT DISC

NOT APPLICABLE

### BACKGROUND OF THE INVENTION

1. Technical Field of the Invention

This invention relates generally to sensors for monitoring atrial fibrillation in a patient.

2. Description of Related Art

An electrocardiograph is a device that monitors electrical impulses generated by a patent's heart via a plurality of electrodes that are generally attached to a patient's skin. The resulting graph of such electrical impulses is referred to as an electrocardiogram (an ECG or EKG). Electrocardiograms are read by medical personnel to detect heart rate as well as to detect irregularities in the heart based on abnormal rhythms.

One such abnormal heart rhythm is atrial fibrillation that is indicated by a plurality of indicators, such as irregular R-wave intervals, tachycardia, and the absence of P-waves in a patient's electrocardiogram. Atrial fibrillation can manifest itself as heart palpitations, fainting, chest pains or even in congestive heart failure, however, many cases of atrial fibrillation present themselves without symptoms. Undetected atrial fibrillation can be a serious due to a greatly increased risk of stroke, with a high risk of such stroke being either debilitating or fatal. Atrial fibrillation can occur in episodes that last minutes, hours or even weeks. Continuous monitoring of a patient's electrocardiogram is required to detect the presence and absence of atrial fibrillation, given the intermittent nature of episodes, however, typical monitoring devices must be strapped to the patient for hours or days and can be intrusive to normal activities.

Other disadvantages of conventional approaches will be evident to one skilled in the art when presented the disclosure that follows.

### BRIEF SUMMARY OF THE INVENTION

The present invention is directed to apparatus and methods of operation that are further described in the following Brief Description of the Drawings, the Detailed Description of the Invention, and the claims. Other features and advantages of the present invention will become apparent from the following detailed description of the invention made with reference to the accompanying drawings.

### BRIEF DESCRIPTION OF THE SEVERAL VIEWS OF THE DRAWING(S)

FIG. 1 is a schematic block diagram of an embodiment of a needle implantable atrial fibrillation monitor 50 and injection needle 10 in accordance with the present invention;

FIG. 2 is a schematic block diagram of an embodiment of a needle implantable atrial fibrillation monitor 50 in accordance with the present invention;

FIG. 3 is a schematic block diagram of an embodiment of a sensing circuit in accordance with the present invention;

FIG. 4 is a schematic block diagram of an embodiment of an interaction between a base station 150 a needle implantable atrial fibrillation monitor 50 in accordance with the present invention;

FIG. 5 is a schematic block diagram of an embodiment of a base station 150 in accordance with the present invention;

FIG. 6 is a pictorial diagram of an embodiment of several screen displays of base station 150 in accordance with the present invention;

FIG. 7 is a schematic block diagram of an embodiment of an interaction between a charging unit 160 and a needle implantable atrial fibrillation monitor 50 in accordance with the present invention;

FIG. 8 is a flowchart representation of an embodiment of a method in accordance with the present invention; and

FIG. 9 is a flowchart representation of an embodiment of a method in accordance with the present invention.

### DETAILED DESCRIPTION OF THE INVENTION

FIG. 1 is a schematic block diagram of an embodiment of a needle implantable atrial fibrillation monitor 50 and injection needle 10 in accordance with the present invention. In particular, a portion of needle 10 is shown that includes a hollow shaft that is sized to fit the needle implantable atrial fibrillation monitor 50. The needle implantable atrial fibrillation monitor 50 has a form factor such as a small rod or cylinder that is 3 mm or less in width and 20-40 mm in length, though other dimensions can likewise be employed.

The implantable atrial fibrillation monitor 50 is injected by piercing the skin of the patient via needle 10 and applying a force to the implantable atrial fibrillation monitor 50 in the direction of the arrow shown to exit the needle 10 and remain implanted beneath the skin of the patient. Force can be applied to the implantable atrial fibrillation monitor 50 via compressed air, via a fluid, such as sterile saline solution or other sterile solution, via a spring-loaded plunger or other force applied in conjunction with the operation of needle 10.

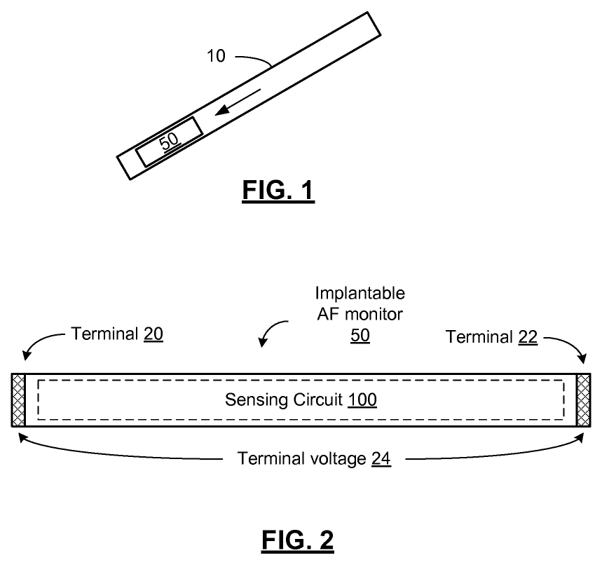

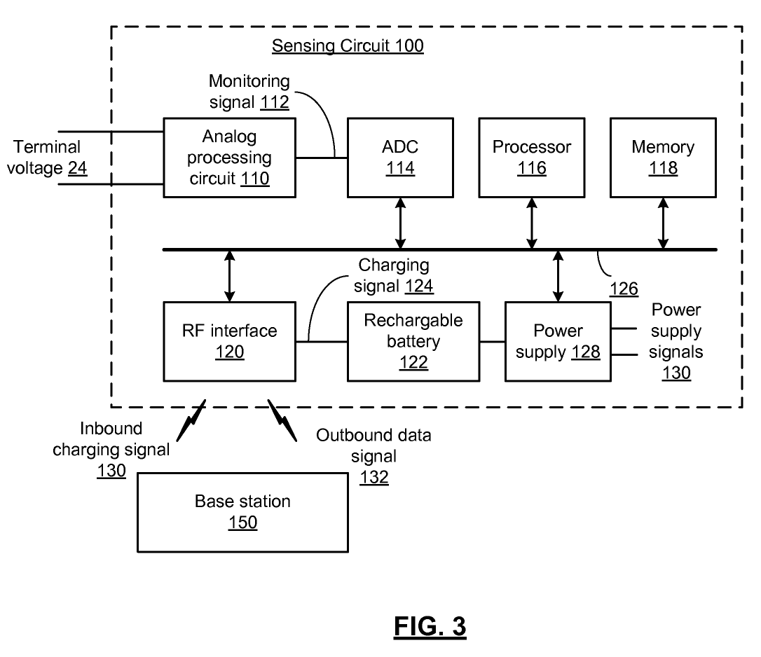

FIG. 2 is a schematic block diagram of an embodiment of a needle implantable atrial fibrillation monitor 50 in accordance with the present invention. In particular, needle implantable atrial fibrillation monitor 50 includes a sensing circuit 100 that is electrically coupled to a plurality of terminals such as terminals 20 and 22. Terminals 20 and 22 are positioned to produce a terminal voltage 24, when implanted subcutaneously in the chest of a patient or other portion of the patient's body, in response to electrical activity of the heart. In particular, the terminal voltage 24 is a weak electrical signal that varies with time in a fashion to represent the patient's electrocardiogram. While the terminals 20 and 22 are shown as rectangular, other shapes such as squares and other polygons, or ovals, can be employed. In an embodiment of the present invention the ends of the needle implantable atrial fibrillation monitor 50 are hemispherical and the terminals 20 and 22 make up the hemispherical ends, however, other shapes conforming to the three-dimensional surface of the needle implantable atrial fibrillation monitor 50 can likewise be employed.

The implantation of the needle implantable atrial fibrillation monitor 50 and operation of the monitor, while implanted, provides less restrictions on the actions of the patient while the monitoring occurs. In this fashion, the patient can more fully participate in normal day to day activities while the needle implantable atrial fibrillation monitor 50 is engaged in monitoring the patient's electrocardiogram. For instance, the needle implantable atrial fibrillation monitor 50 can employ a housing that is somewhat flexible so as to flex with the movement of the patient's body.

Further details regarding the operation of the needle implantable atrial fibrillation monitor 50, and in particular the communication of data from the needle implantable atrial fibrillation monitor 50 to a base station, will be described in greater detail in conjunction with FIGS. 3-8, which further describe several optional functions and features.

FIG. 3 is a schematic block diagram of an embodiment of a sensing circuit 100 in accordance with the present invention. In particular, sensing circuit 100 includes an analog processing circuit 110 that generates a monitoring signal 112 based on the terminal voltage 24. In an embodiment of the present invention, analog processing circuit 110 includes an instrumentation differential amplifier, cascaded with as a low noise gain amplifier with optional filtration, for amplifying the terminal voltage 24 and filtering out electrical signals that do not fall within the expected frequency range for an electrocardiogram. Additionally the entire analog path can be duty-cycled, since the low frequency nature of the electrocardiogram signal and the fact that the analog subsystem only needs to be active at the time when a sample is being captured.

Analog to digital converter 114, such as a successive approximation converter, sigma-delta modulator or other analog to digital converter, converts the monitoring signal 112 to monitoring data. Much like the analog subsystem, the ADC can be powered when a sample needs to be captured.

Processor 116 compresses the monitoring data and generates compressed data in response thereto for storage in memory 118. The compression can be accomplished in several different ways. In one embodiment of the present invention, the monitoring data can be compressed using a discrete cosine transform (DCT) based approach. Additionally, in order to further increase the compression ratio, the QRS complex can be identified, tagged and removed from the waveform; the resulting signal is then compressed with a DCT approach. The base station 150 or a remote reporting station in communication therewith can, in turn, use the compressed signal and the QRS complex tag to reconstruct the original signal.

In addition or in the alternative, features of the monitoring data, such as P waves, QRS complex and T waves can be identified, and characterized and used to recreate or characterize the monitoring data that makes up the electrocardiogram. In a further embodiment of the present invention, the processor 116 detects an anomaly in the monitoring data and produces compressed data that includes event data that indicates the anomaly. For instance, the processor 116 can detect an atrial fibrillation event by an absence of P waves and generate event data corresponding to the number of occurrences, and duration of an atrial fibrillation event or events. Another additional mode of operation is that the processor 116 starts recoding event data when notified by a patient, assuming that he or she is experiencing some symptoms; such trigger will be generated from the base station 150 via the RF interface 120.

Processor 116 can be implemented using a shared processing device, individual processing devices, or a plurality of processing devices and may further include memory. Such a processing device may be a microprocessor, micro-controller, digital signal processor, microcomputer, central processing unit, field programmable gate array, programmable logic device, state machine, logic circuitry, digital circuitry, and/or any device that manipulates signals based on operational instructions. The memory may be a single memory device or a plurality of memory devices. Such a memory device may be a read-only memory, random access memory, volatile memory, non-volatile memory, static memory, dynamic memory, flash memory, and/or any device that stores digital information. Note that when the processor 116 implements one or more of its functions via a state machine, digital circuitry, and/or logic circuitry, the memory storing the corresponding operational instructions is embedded with the circuitry comprising the state machine, digital circuitry, and/or logic circuitry. While a particular bus architecture is shown that includes bus 126, other architectures with additional direct connectivity between modules or with additional buses are likewise possible.

RF interface 120 includes a transceiver and antenna to transmit the compressed data to a base station 150 via RF signaling, such as outbound data signal 132. In particular, RF interface 120 operates at an RF frequency in the range from 100 KHz up to 5 GHz or more. The RF interface 120 is designed to operate when the needle implantable atrial fibrillation monitor is implanted in the patient's body. In an embodiment of the present invention, the RF interface 120 is implemented using RFID technology with the sensing circuit operating as an RFID tag and with the remote base station 150 operating as an RFID reader. For example, sensing circuit 100 can include a rechargeable battery 122 and a power supply 128 that is coupled to supply one or more power supply signal 130 to the analog processing circuit 110, the analog to digital converter 114, the processor 116 and the memory 118. The power supply 128 includes one or more voltage regulators for generating power supply signals 130 such as an analog voltage and/or a digital voltage. In addition, power supply 128 generates power supply data that indicates the level of charge and/or remaining charge of rechargeable battery 122 that is supplied via bus 126 to processor 116. In this fashion, the power supply data can be included in outbound data signal 132 that is sent to base station 150.

For example, the RF interface 120 can operate as a passive RFID tag that generates a charging signal 124 for recharging the rechargeable battery 122, based on an inbound charging signal 130 received from the base station. In one embodiment this is accomplished by rectifying the RF signal, boosting the DC voltage to a higher value and using either a linear or a switching battery charger to recharge the battery. Further RF interface 120 can generate outbound data 132 based on a backscattering of the inbound charging signal 130.

Other configurations of RF interface 120 are possible are likewise possible with inbound charging signal 130 being at a separate frequency from outbound data signal and/or implemented with a separate RF transceiver. One such embodiment will be presented in conjunction with FIG. 7. In alternative embodiments, rechargeable battery 122 can be charged based on the terminal voltage 24, based on a thermal differential produced by the body, etc.

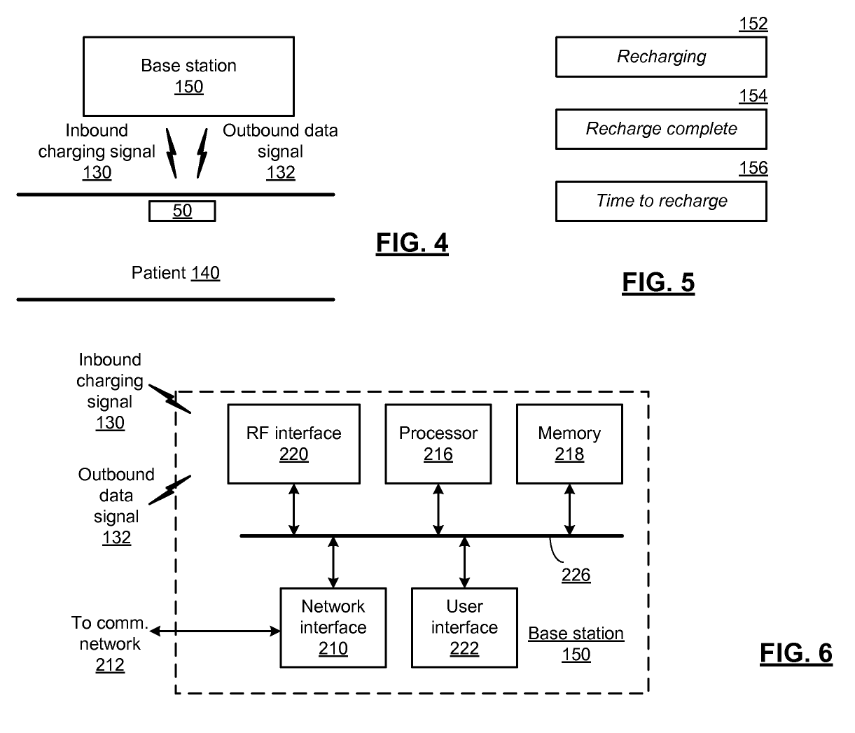

FIG. 4 is a schematic block diagram of an embodiment of an interaction between a base station 150 a needle implantable atrial fibrillation monitor 50 in accordance with the present invention. In particular, the operation of needle implantable atrial fibrillation monitor 50 can be described in conjunction with the following example. The needle implantable atrial fibrillation monitor 50 is implanted beneath the skin of the chest, arm, leg or other portion of the patient 140. The rechargeable battery 122 was charged prior to the injection of the needle implantable atrial fibrillation monitor 50 and is capable of operating the needle implantable atrial fibrillation monitor 50 for 18 hours, 24 hours, 36 hours or several days or weeks without being recharged. In this fashion the patient can go about normal activities as their health allows with the needle implantable atrial fibrillation monitor 50 operating on a periodic, continuous or substantially continuous basis to generate a monitoring signal based on the terminal voltage 24 that is converted to monitoring data and compressed for storage in memory 118. In particular, the patient 140 need not be in constant range of base station 150, the charge of rechargeable battery 122 is capable of maintaining the operation of the needle implantable atrial fibrillation monitor 50 for extended periods of time. In addition the battery can entirely run out during a period where monitoring may not be needed; but if subsequently a monitoring operation becomes necessary, the system will allow the fully depleted battery to be recharged and normal operations to resume.

Base station 150 can be incorporated in a device that is placed near the bed of patient 140, on a night stand, on an office desk or near a chair where patient 140 sits for periods of time or otherwise placed in proximity to the patient 140 periodically such as once a day, to download the compressed data via outbound data signal 132. In this fashion, compressed data reflecting the complete history of the patient 140 (since the last download) can be downloaded to the base station 150 for later review and analysis by medical personnel. By repeated downloads of compressed data, the base station can compile compressed data that indicates an electrocardiogram for the patient on a 24 hours a day, seven days a week, as long as the needle implantable atrial fibrillation monitor 50 remains implanted.

FIG. 5 is a pictorial diagram of an embodiment of several screen displays of base station 150 in accordance with the present invention. In particular base station 150 includes a screen display, such as an liquid crystal display, touch screen or other display devices that generates screen displays 152, 154 and 156 to indicate the charging status of needle implantable atrial fibrillation monitor 50.

As shown in screen display, 152, base station 150 provides an indication when the base station 150 is in range of needle implantable atrial fibrillation monitor 50 and charging of the device is in progress. As discussed in conjunction with FIG. 3, outbound data signal 132 can include power supply data that indicates the level of charge of the rechargeable battery 122 and base station 150 can monitor and indicate the level of charge to the patient. When charging is complete as indicated by the power supply data, base station 150 can indicate this to the patient 140 via an indication such as screen display 154. Base station can cease charging at this time or continue charging needle implantable atrial fibrillation monitor 50 as long as needle implantable atrial fibrillation monitor 50 is in range. However, the screen display 154 can indicate to the patient 140 that they are free to move away from the base station 150 if they desire.

In addition, base station 150 can be preprogrammed with the time period of operation of the needle implantable atrial fibrillation monitor 50 on a single charge of rechargeable battery 122. After charging ends, either because the needle implantable atrial fibrillation monitor 50 has gone out of range or the base station 150 has ceased charging of the needle implantable atrial fibrillation monitor 50, the base station 150 can count down to the time when the needle implantable atrial fibrillation monitor 50 should be recharged again, allowing for a certain safety period allowing for a margin of error and for a reasonable the period of time for the patient to react to the alert. In response, base station 150 can generate an indication such as screen display 156 indicating that it is time to recharge the needle implantable atrial fibrillation monitor 50, optionally accompanied by an audible indicator such as an audible alarm. In this fashion, the patient 140 can be alerted to move within range of the base station 150 for recharging.

In a further embodiment of the present invention, the base station 150 can also keep track of the last time that the compressed data was downloaded from the needle implantable atrial fibrillation monitor 50 and estimate the amount of remaining unused memory, and generate an indication and/or alarm when the needle implantable atrial fibrillation monitor 50 is running low of memory so that the patient can be alerted to move within range of the base station 150 for downloading the stored data.

While screen displays 152, 154 and 156 indicate the charging status of needle implantable atrial fibrillation monitor 50, it should be noted that other user interface options, patient alerts or other functions can likewise be implemented via such screen displays. Further, while the charging status of needle implantable atrial fibrillation monitor 50 is shown in conjunction with screen displays, other user interface devices such as audible indicators or alarms, light emitting diodes of other user interface devices can likewise be employed.

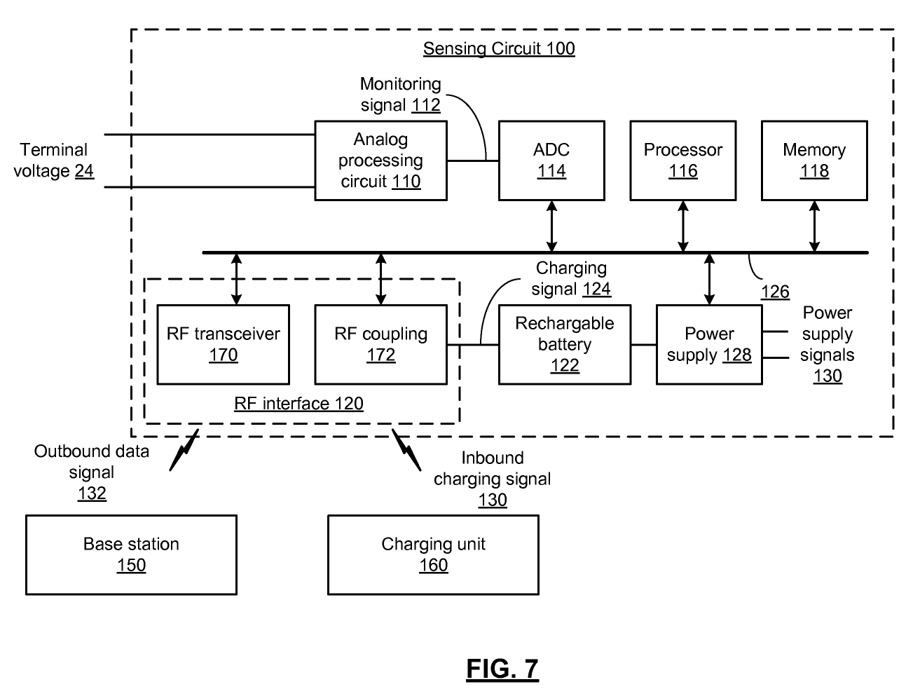

FIG. 6 is a schematic block diagram of an embodiment of a base station 150 in accordance with the present invention. In particular, base station 150 includes an RF interface 220, processor 216, memory 218, network interface 210 and user interface 222 that are coupled via bus 226.

RF interface 220 can be implemented via an RFID reader or other RF transceiver and antenna that are complementary to RF transceiver 120 to facilitate the reception of outbound data signal 132 and the generation of inbound charging signal 130. Processor 216 executes a base station application that directs the performance of the various functions and features of base station 150. Processor 216 can be implemented using a shared processing device, individual processing devices, or a plurality of processing devices and may further include memory. Such a processing device may be a microprocessor, micro-controller, digital signal processor, microcomputer, central processing unit, field programmable gate array, programmable logic device, state machine, logic circuitry, digital circuitry, and/or any device that manipulates signals based on operational instructions. The memory may be a single memory device or a plurality of memory devices. Such a memory device may be a read-only memory, random access memory, volatile memory, non-volatile memory, static memory, dynamic memory, flash memory, and/or any device that stores digital information. Note that when the processor 216 implements one or more of its functions via a state machine, digital circuitry, and/or logic circuitry, the memory storing the corresponding operational instructions is embedded with the circuitry comprising the state machine, digital circuitry, and/or logic circuitry. While a particular bus architecture is shown that includes bus 226, other architectures with additional direct connectivity between modules or with additional buses are likewise possible.

As discussed in conjunction with FIG. 4, base station 150 downloads the compressed data from the needle implantable atrial fibrillation monitor 50 and stores the compressed data in memory 218. In an embodiment of the present invention, memory 218 includes a removable memory device such as a memory card, memory stick, removable disk or other removable memory device that can store one or more downloads of the compressed data received from the needle implantable atrial fibrillation monitor 50 to be supplied to medical personnel for analysis. Further, base station 150 includes a network interface, such as a dial-up modem, broadband modem, 802.11 interface or other network interface to couple the base station 150 to a communication network such as the Internet. In this fashion, the compressed data downloaded from the needle implantable atrial fibrillation monitor 50 can be uploaded to a remote reporting station coupled to the communication network 212, such as a medical server, a physician's computer or other remote station for analysis.

User interface 222 includes a display device for generating screen displays such as screen displays 152, 164 and 156. In addition, user interface optionally includes user input devices such as a touch screen, one or more buttons, an audible alarm and/or other user interface devices to be used during the setup of the base station 150, the registration and/or pairing of the base station 150 with the needle implantable atrial fibrillation monitor 50, the registration and/or pairing of the base station 150 with a remote reporting station, the set-up of communication via a communication network 212 and other functions and features.

FIG. 7 is a schematic block diagram of an embodiment of an interaction between a charging unit 160 and a needle implantable atrial fibrillation monitor 50 in accordance with the present invention. In particular, a needle implantable atrial fibrillation monitor 50 is presented that includes similar elements to the embodiment of FIG. 3 that are referred to by common reference numerals. In this embodiment, RF interface 120 includes a standard RF transceiver 170 for communicating bidirectionally with a complementary transceiver of base station 150. In addition, RF interface 120 includes a separate RF coupling, such as a near field coil antenna and rectifier circuit for converting an inbound charging signal 130 from charging unit 160 into charging signal 124.

Charging unit 160 can include an RF generator and near field coil antenna or other RF coupling to transfer power, magnetically or electromagnetically from the charging unit 160 to the implant. In an embodiment of the present invention, the charging unit is a small battery-power device that, during charging of the needle implantable atrial fibrillation monitor 50, is worn in close proximity to the implant using a chest strap or a similar garment. In another embodiment, a disposable solution can be employed, which consists of a sticking patch that includes a button battery, an inductive coil and the circuitry necessary to recharge the system; such patch can be worn underneath the ordinary clothing for the amount of time necessary to recharge the implant.

In various embodiments, the charging unit 160 and base station 150 can be combined. In this case it may be advantageous to implement inbound charging signal 130 with longer range RF signaling so that base station can be spaced several feet from base station 150/charging unit 160 during charging. In this instance, alternative antenna configurations for RF coupling 172 can be employed. It should also be noted that RF transceiver 170 and RF coupling can be implemented at different frequencies with separate antennas/coils, with a single multifrequency antenna/coil or at a single frequency with a single antenna/coil. Further, communication with the base station 150 and charging via charging unit 160 can be performed at separate times or contemporaneously, whether or not the functionality of charging unit 160 and base station 150 are combined

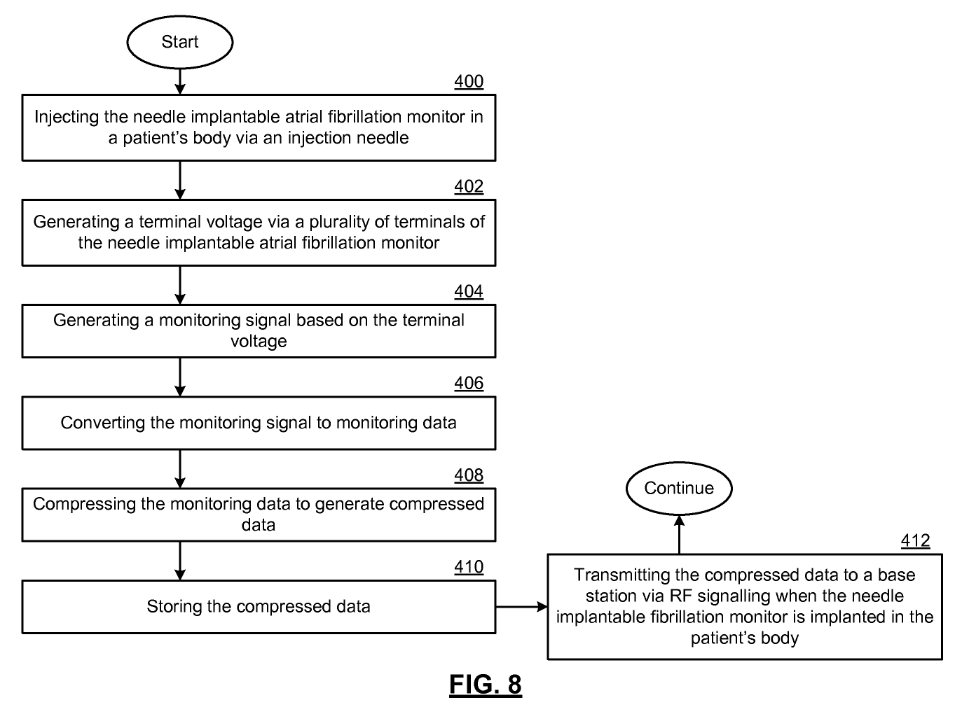

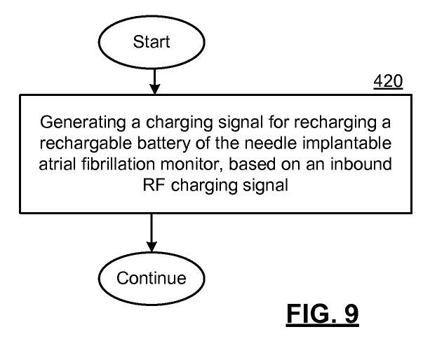

FIG. 8 is a flowchart representation of an embodiment of a method in accordance with the present invention. In particular, a method is presented for use in conjunction with one or more features and functions described in conjunction with FIGS. 1-6. In step 400, the needle implantable atrial fibrillation monitor is injected in a patient's body via an injection needle. In step 402, a terminal voltage is generated via a plurality of terminals of the needle implantable atrial fibrillation monitor. In step 404, a monitoring signal is generated based on the terminal voltage. In step 406, the monitoring signal is converted to monitoring data. In step 408, the monitoring data is compressed to generate compressed data. In step 410, the compressed data is stored. In step 412, the compressed data is transmitted to a base station via RF signaling when the needle implantable atrial fibrillation monitor is implanted in the patient's body.

In an embodiment of the present invention, step 408 includes detecting an anomaly in the monitoring data and wherein the compressed data includes event data that indicates the anomaly. The anomaly can include an atrial fibrillation event. Step 404 can generate the monitoring signal continuously over a 24 hour period or longer.

FIG. 9 is a flowchart representation of an embodiment of a method in accordance with the present invention. In particular, a method is presented for use in conjunction with one or more features and functions described in conjunction with FIGS. 1-7. In step 420, a charging signal is generated for recharging a rechargeable battery of the needle implantable atrial fibrillation monitor, based on an inbound RF charging signal.

As may be used herein, the terms “substantially” and “approximately” provides an industry-accepted tolerance for its corresponding term and/or relativity between items. Such an industry-accepted tolerance ranges from less than one percent to fifty percent and corresponds to, but is not limited to, component values, integrated circuit process variations, temperature variations, rise and fall times, and/or thermal noise. Such relativity between items ranges from a difference of a few percent to magnitude differences. As may also be used herein, the term(s) “coupled to” and/or “coupling” and/or includes direct coupling between items and/or indirect coupling between items via an intervening item (e.g., an item includes, but is not limited to, a component, an element, a circuit, and/or a module) where, for indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”. As may even further be used herein, the term “operable to” indicates that an item includes one or more of power connections, input(s), output(s), etc., to perform one or more its corresponding functions and may further include inferred coupling to one or more other items. As may still further be used herein, the term “associated with”, includes direct and/or indirect coupling of separate items and/or one item being embedded within another item.

The present invention has also been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claimed invention.

The present invention has been described above with the aid of functional building blocks illustrating the performance of certain significant functions. The boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality. To the extent used, the flow diagram block boundaries and sequence could have been defined otherwise and still perform the certain significant functionality. Such alternate definitions of both functional building blocks and flow diagram blocks and sequences are thus within the scope and spirit of the claimed invention. One of average skill in the art will also recognize that the functional building blocks, and other illustrative blocks, modules and components herein, can be implemented as illustrated or by discrete components, application specific integrated circuits, processors executing appropriate software and the like or any combination thereof.

The present invention has been described in conjunction with various illustrative embodiments that include many optional functions and features. It will be apparent to those skilled in the art that the disclosed invention may be modified in numerous ways, the functions and features of these embodiments can be combined in other embodiments not expressly shown, and may assume many embodiments other than the preferred forms specifically set out and described above. Accordingly, it is intended by the appended claims to cover all modifications of the invention which fall within the true spirit and scope of the invention.

What is claimed is:

1. A needle implantable atrial fibrillation monitor comprising:
a plurality of terminals that generate a terminal voltage when the needle implantable atrial fibrillation monitor is implanted in a patient's body;
an analog processing circuit, coupled to the plurality of terminals, that generates a monitoring signal based on the terminal voltage;
an analog to digital converter, coupled to the analog processing circuit, that converts the monitoring signal to monitoring data;
a processor, coupled to the analog to digital converter, that analyzes the monitoring data, detects events and that generates compressed event data in response thereto;
a memory, coupled to the processor, for storing the compressed data; and
an RF interface, coupled to the memory, that transmits the compressed data to a base station via RF signaling when the needle implantable atrial fibrillation monitor is implanted in the patient's body

2. The needle implantable atrial fibrillation monitor of claim 1 wherein the processor detects an anomaly in the monitoring data and wherein the compressed data includes event data that indicates the anomaly.

3. The needle implantable atrial fibrillation monitor of claim 1 wherein the anomaly includes an atrial fibrillation event.

4. The needle implantable atrial fibrillation monitor of claim 1 wherein the analog processing circuit generates the monitoring signal continuously over a 24 hour period.

5. The needle implantable atrial fibrillation monitor of claim 1 further comprising:
a rechargeable battery;
a power supply, coupled to the rechargeable battery and coupled to supply at least one power supply signal to the analog processing circuit, the analog to digital converter, the processor and the memory;
wherein the RF interface is further coupled to generate a charging signal for recharging the rechargeable battery, based on an inbound charging signal.

6. A method for use in a needle implantable atrial fibrillation monitor, the method comprising:
injecting the needle implantable atrial fibrillation monitor in a patient's body via an injection needle;
generating a terminal voltage via a plurality of terminals of the needle implantable atrial fibrillation monitor;
generating a monitoring signal based on the terminal voltage;
converting the monitoring signal to monitoring data;
compressing the monitoring data to generate compressed data;
storing the compressed data; and
transmitting the compressed data to a base station via RF signaling when the needle implantable atrial fibrillation monitor is implanted in the patient's body.

7. The method of claim 6 wherein compressing the monitoring data includes:
detecting an anomaly in the monitoring data and wherein the compressed data includes event data that indicates the anomaly.

8. The method of claim 6 wherein the anomaly includes an atrial fibrillation event.

9. The method of claim 6 wherein the monitoring signal is generated continuously over a 24 hour period.

10. The method of claim 6 further comprising:
generating a charging signal for recharging a rechargeable battery of the needle implantable atrial fibrillation monitor, based on an inbound RF charging signal.
